(19) United States
(12) Patent Application Publication
(10) Pub. No.: US 2014/0100457 A1
(43) Pub. Date: Apr. 10, 2014

(54) TREATMENT INSTRUMENT FOR ENDOSCOPE

(71) Applicant: OLYMPUS MEDICAL SYSTEMS CORP., Tokyo (JP)

(72) Inventors: Kenichi NISHINA, Tokyo (JP); Atsushi BAN, Tokyo (JP)

(73) Assignee: OLYMPUS MEDICAL SYSTEMS CORP., Tokyo (JP)

(21) Appl. No.: 14/048,417

(22) Filed: Oct. 8, 2013

Related U.S. Application Data
(63) Continuation of application No. PCT/JP2013/060623, filed on Apr. 8, 2013.

(30) Foreign Application Priority Data
May 10, 2012 (JP) 2012-108754

(51) Int. Cl. A61B 10/04; A61B 8/12; A61B 8/00

(52) U.S. Cl. CPC A61B 10/04; A61B 8/445; A61B 8/12
USPC 600/437; 600/570

(57) ABSTRACT

A treatment instrument for an endoscope includes a specimen sampling tool including arm portions, a loading portion, and a claw portion, a proximal end of the arm portions being fixed, and a tube on an inside of which the specimen sampling tool is placed to be capable of moving back and forth. At least the claw portion is expanded in diameter further to an outer side in a radial direction R than a tube 3 according to relative movement of the specimen sampling tool and the tube 3. After the diameter expansion, the arm portions are retracted in the tube according to relative movement of the specimen sampling tool and the tube opposite to the relative movement in the diameter expansion in the extending direction.

### CROSS REFERENCE TO RELATED APPLICATION

This application is a continuation application of PCT/JP2013/060623 filed on Apr. 8, 2013 and claims benefit of Japanese Application No. 2012-108754 filed in Japan on May 10, 2012, the entire contents of which are incorporated herein by this reference.

### BACKGROUND OF THE INVENTION

1. Field of the Invention

The present invention relates to a treatment instrument for an endoscope including a specimen sampling tool for sampling cells or tissues on an inner peripheral surface of a tubular subject.

2. Description of the Related Art

A method is well known wherein after inserting an insertion portion of an endoscope to a vicinity of a small-diameter duct, into which the insertion portion of the endoscope cannot be inserted, such as a lung periphery of a bronchus, a bile duct, or a pancreatic duct in a subject, a tube provided with an X-ray marker on a distal end side is inserted into the small-diameter duct, to a vicinity of an examined region in the small-diameter duct under X-ray observation via a treatment instrument channel provided in the insertion portion, and, thereafter, a specimen sampling tool for sampling such as forceps or a puncture needle is inserted into the tube to sample cells or tissues of the examined region.

Cells or tissues of the examined region in a position opposed to a distal end of the tube in the duct can be sampled by the forceps or the puncture needle. However, since a space for changing a direction of the specimen sampling tool projecting from the tube distal end is absent in the small-diameter duct, there is a problem in that it is difficult to sample cells or tissues of the examined region located on an inner peripheral surface of the duct opposed to an outer peripheral surface of the tube.

Therefore, Japanese Patent Application Laid-Open Publication No. 2001-269345 discloses a specimen sampling tool provided with a brush at a distal end of a wire that can be inserted into a tube and can scrape off a cell on an inner peripheral surface of a duct.

### SUMMARY OF THE INVENTION

A treatment instrument for an endoscope includes: a specimen sampling tool including at least two arm portions having flexibility, loading portions provided at a distal end in an extending direction of the arm portions, a collected specimen from a subject being loaded on the loading portion, and claw portions provided at the distal end of the arm portions, formed to be bent to a loading surface side of the loading portion, on which the collected specimen is loaded, to include the loading portion on an inside, and configured to shave the subject, a proximal end in the extending direction of the arm portions being bound and fixed to the specimen sampling tool; and a tube on an inside of which the specimen sampling tool is placed to be capable of moving back and forth in the extending direction. At least the claw portions in the arm portions is expanded in diameter further to an outer side in a radial direction of the tube than the tube according to relative movement of the specimen sampling tool and the tube in the extending direction and, as a result of the diameter expansion, a plurality of the loading surfaces and a plurality of the claw portions respectively face outward with respect to an axis of the specimen sampling tool. After the diameter expansion, the arm portions are retracted in the tube according to relative movement of the specimen sampling tool and the tube opposite to the relative movement in the diameter expansion in the extending direction.

### BRIEF DESCRIPTION OF THE DRAWINGS

FIG. 1 is a perspective view showing a treatment instrument for an endoscope in a first embodiment.

FIG. 2 is a partial sectional view of the treatment instrument for an endoscope taken along line II-II in FIG. 1.

FIG. 3 is a partial sectional view showing a state in which arm portions of a rod for cell sampling in a specimen sampling tool shown in FIG. 2 are further expanded in diameter than a tube.

FIG. 4 is a partial sectional view schematically showing a method of specifying an inclination angle of a through-hole of the tube shown in FIG. 2.

FIG. 5 is a partial perspective view showing in enlargement a distal end side of the arm portions shown in FIG. 2.

FIG. 6 is a partial perspective view showing a modification of a shape of a claw portion shown in FIG. 5.

FIG. 7 is a perspective view showing a modification of the treatment instrument for an endoscope shown in FIG. 1.

FIG. 8 is a partial sectional view of the treatment instrument for an endoscope taken along line VI-VI in FIG. 7.

FIG. 9 is a partial sectional view showing a state in which arm portions of a rod for cell sampling in a specimen sampling tool shown in FIG. 8 are further expanded in diameter than a tube.

FIG. 10 is a partial sectional view of a treatment instrument for an endoscope in a second embodiment taken along line VIII-VIII in FIG. 1.

FIG. 11 is a partial sectional view showing a state in which arm portions of a rod for cell sampling in a specimen sampling tool shown in FIG. 10 are further expanded in diameter than a tube.

FIG. 12 is a partial sectional view of the treatment instrument for an endoscope taken along line X-X in FIG. 7.

FIG. 13 is a partial sectional view showing a state in which the arm portions of the rod for cell sampling in the specimen sampling tool shown in FIG. 12 are further expanded in diameter than the tube.

FIG. 14 is a diagram schematically showing an endoscope system including the treatment instrument for an endoscope shown in FIG. 1.

### DETAILED DESCRIPTION OF THE PREFERRED EMBODIMENTS

Embodiments of the present invention are explained below with reference to the drawings.

### First Embodiment

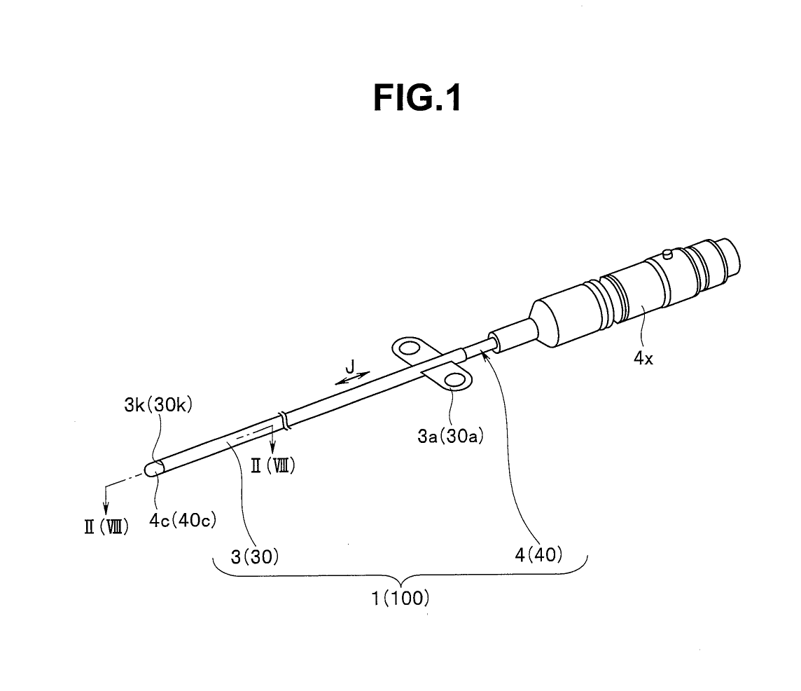

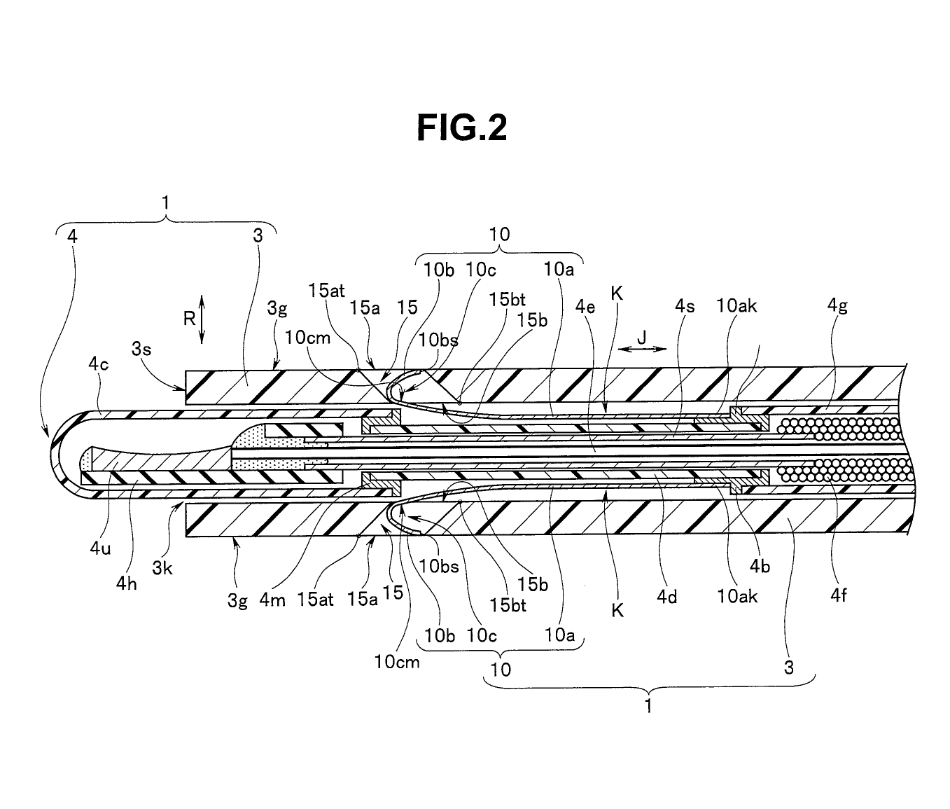

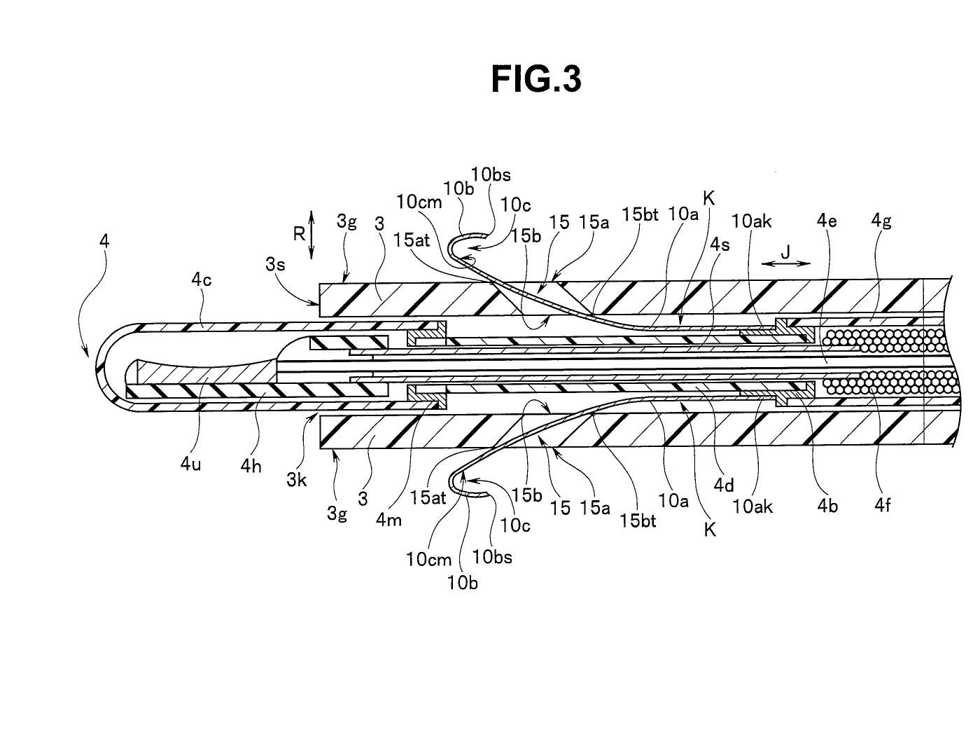

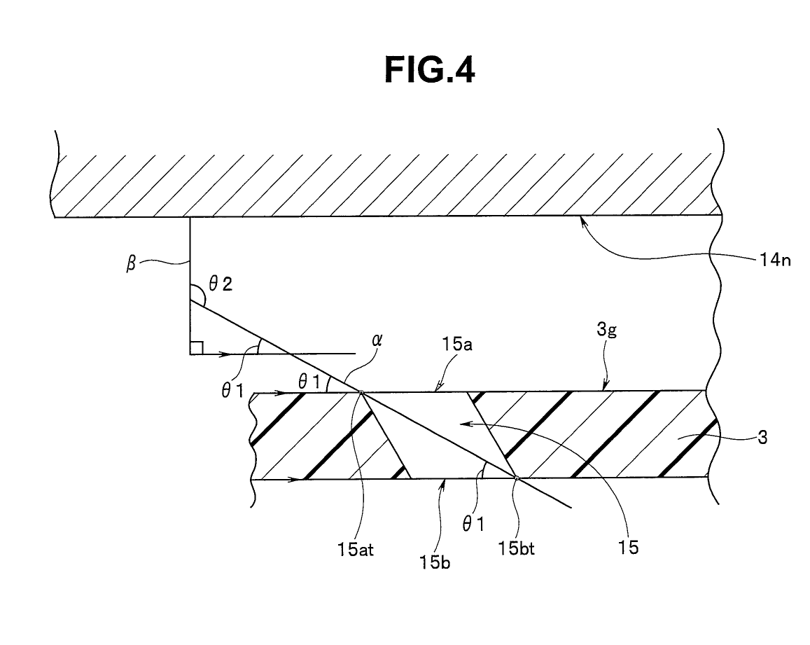

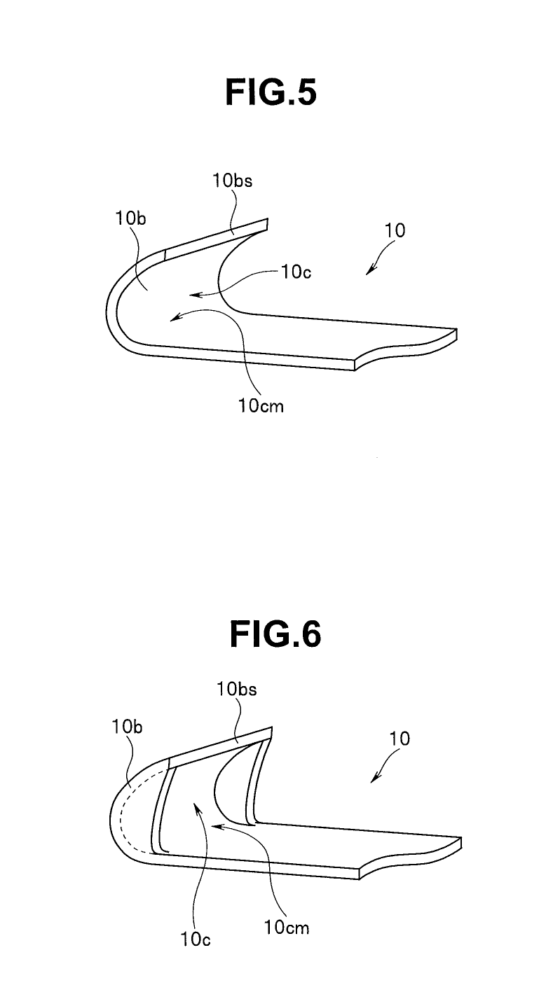

FIG. 1 is a perspective view showing a treatment instrument for an endoscope in a first embodiment. FIG. 2 is a partial sectional view of the treatment instrument for an endoscope taken along line II-II in FIG. 1. FIG. 3 is a partial sectional view showing a state in which arm portions of a rod for cell sampling in a specimen sampling tool shown in FIG. 2 are further expanded in diameter than a tube. FIG. 4 is a partial sectional view schematically showing a method of specifying an inclination angle of a through-hole of the tube shown in FIG. 2. FIG. 5 is a partial perspective view showing in enlargement a distal end side of the arm portions shown in FIG. 2. FIG. 6 is a partial perspective view showing a modification of a shape of a claw portion shown in FIG. 5.

As shown in FIGS. 1 and 2, a main part of a treatment instrument for an endoscope 1 includes a tube 3 and a specimen sampling tool 4.

The tube 3 is formed to be elongated along an extending direction J of the specimen sampling tool 4. As shown in FIGS. 1 and 2, the specimen sampling tool 4 is capable of moving back and forth in the extending direction J on an inside of the tube 3.

The tube 3 is inserted into a subject having a plurality of bent portions. Therefore, in order to secure insertability, the tube 3 is formed of resin having flexibility and having biocompatibility. Note that examples of the resin forming the tube 3 include polyethylene, fluororesin, and PEEK.

As shown in FIG. 1, on a proximal end side in the extending direction J of the tube 3, an operation portion 3a for rotating the tube 3 in a circumferential direction of the tube 3 and moving the tube 3 back and forth in the extending direction J is provided.

Note that, in the present embodiment, the specimen sampling tool 4 is configured with an ultrasound probe elongated along the extending direction J. More specifically, the specimen sampling tool 4 is configured with a mechanical radial scanning probe.

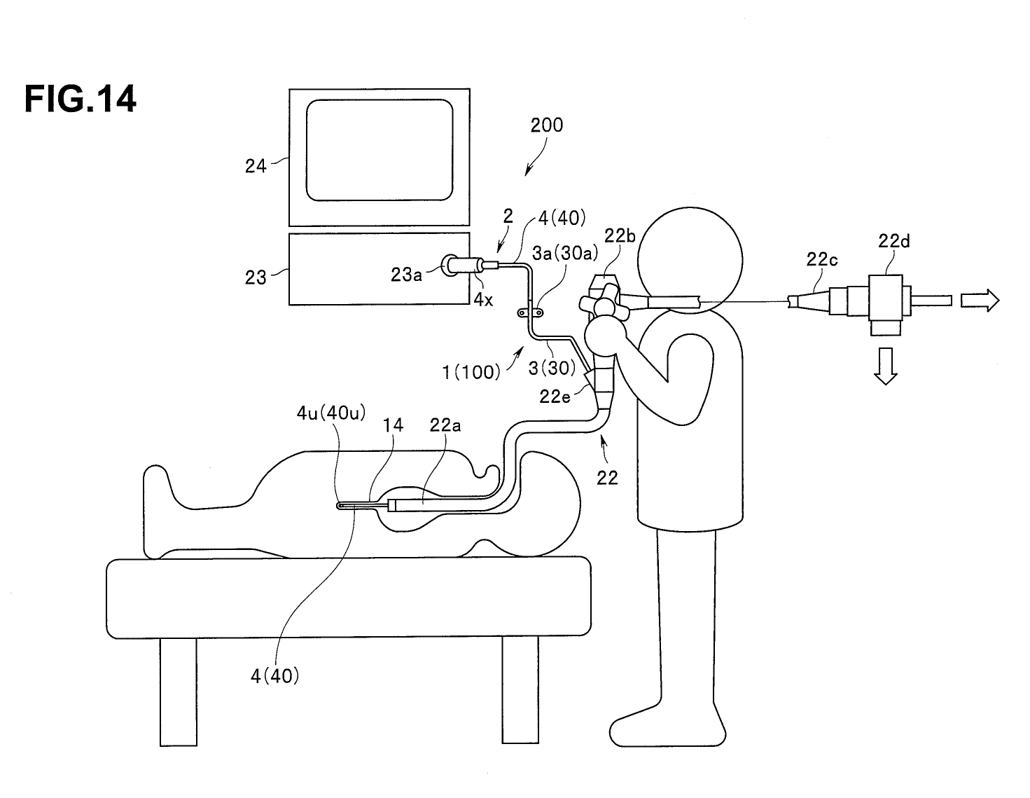

More specifically, the specimen sampling tool 4 includes an ultrasound observation portion 4u located at a distal end in the extending direction J and configured with a single ultrasound transducer of a single plate and a housing 4h configured to hold the ultrasound observation portion 4u. The specimen sampling tool 4 includes, toward a rear in the extending direction J from the ultrasound observation portion 4u, an ultrasound transducer cable 4e extended to a below-mentioned connector 4x (see FIG. 1) and configured to transmit an electric pulse signal from a below-mentioned ultrasound observation device 23 (see FIG. 14) to the ultrasound observation portion 4u.

The specimen sampling tool 4 includes a small-diameter shaft 4s including the ultrasound transducer cable 4e and extended backward in the extending direction J from the housing 4h. Further, the specimen sampling tool 4 includes a flexible shaft 4f including the ultrasound transducer cable 4e, fixed with a proximal end in the extending direction J of the small-diameter shaft 4s, and configured to apply rotation power to the housing 4h via the small-diameter shaft 4s.

Further, the specimen sampling tool 4 includes a sheath large-diameter portion 4g covering an outer periphery of the flexible shaft 4f, a cap 4b fixed to a distal end in the extending direction J of the sheath large-diameter portion 4g, and a sheath small-diameter portion 4d covering an outer periphery of the small-diameter shaft 4s and having a proximal end in the extending direction J fixed to the cap 4b. The specimen sampling tool 4 includes a cap 4m fixed to a proximal end in the extending direction J of the sheath small-diameter portion 4d and a distal end cap 4c covering the ultrasound observation portion 4u held by the housing 4h and having a distal end in the extending direction J fixed to the cap 4m. The specimen sampling tool 4 includes a rod for cell sampling 10.

That is, a main part of the specimen sampling tool 4 includes the ultrasound observation portion 4u, the housing 4h, the ultrasound transducer cable 4e, the small-diameter shaft 4s, the flexible shaft 4f, the sheath large-diameter portion 4g, the cap 4b, the sheath small-diameter portion 4d, the cap 4m, the distal end cap 4c, and the rod for cell sampling 10. The specimen sampling tool 4 is detachably attachable to the below-mentioned ultrasound observation device 23 (see FIG. 14) via the connector 4x provided at a proximal end in the extending direction J.

In the specimen sampling tool 4, according to relative movement with the tube 3 in the extending direction J, at least the ultrasound observation portion 4u is capable of projecting further forward in the extending direction J than the distal end 3s via an opening 3k formed at the distal end 3s in the extending direction J of the tube 3.

The sheath small-diameter portion 4d is formed in a small diameter further in a radial direction R of the specimen sampling tool 4 than the sheath large-diameter portion 4g and the distal end cap 4c, whereby, in a vicinity of an outer periphery of the sheath small-diameter portion 4d, a space K recessed in the radial direction R is formed between the cap 4m and the cap 4b along the extending direction J. The rod for cell sampling 10 is located in the space K.

More specifically, a main part of the rod for cell sampling 10 includes at least two arm portions 10a having flexibility and located along the extending direction J, claw portions 10b provided at distal ends in the extending direction J of the respective arm portions 10a, distal ends of the claw portions 10b shaving cells or tissues on an inner peripheral surface 14n (see FIG. 4) of a subject, and loading portions 10c provided in bent portions in the claw portions 10b, cells or tissues, which are collected specimens shaved by the claw portions 10b, being loaded on the loading portions 10c. In the rod for cell sampling 10, proximal ends 10ak in the extending direction J of the arm portions 10a are bound and fixed to the cap 4b by bonding, welding, brazing, or the like.

The rod for cell sampling 10 is configured of a material having predetermined hardness, having elasticity, and having biocompatibility, for example, a tabular member of a nickel titanium alloy, stainless steel for spring, or the like.

The claw portions 10b are formed to be bent to a loading surfaces 10cm side. As an example, the claw portions 10b are formed to be bent to an outer side in the radial direction R of the arm portions 10a such that an angle formed between the claw portions 10b and the arm portions 10a is equal to or smaller than 90°. Therefore, the claw portions 10b is formed in, for example, a hook shape.

More specifically, as shown in FIG. 5, the claw portion 10b may be formed by bending a flat plate or, as shown in FIG. 6, the claw portion 10b may be formed in a box shape having sidewalls, for example, a shape like a basket of a digger by punching or the like. The claw portion 10b is not limited to be bent and may be formed to be folded. Further, the claw portion 10b may be formed integrally with the arm portion 10a or may be formed separately from the arm portion 10a via, for example, a link member.

The claw portion 10b is located in a linear through-hole 15 formed in an outer peripheral part on a distal end side in the extending direction J of the tube 3 and tilting such that an opening 15b on an inner side of the radial direction R is located further on a proximal end side in the extending direction J than an opening 15a on an outer side in the radial direction R. The claw portion 10b is capable of being retracted in the through-hole 15 from the outside of the tube 3.

Note that, as shown in FIG. 2, in a state in which the claw portions 10b are retracted in the through-hole 15, the distal end side in the extending direction J of the tube 3 is located to overlap the ultrasound observation portion 4u in the extending direction J.

The claw portions 10b are expanded further to an outer side in the radial direction R than an outer peripheral surface 3g of the tube 3 according to relative movement of the specimen sampling tool 4 and the tube 3 in the extending direction J, that is, whether the tube 3 is moved backward in the extending direction J with respect to the specimen sampling tool 4 or the specimen sampling tool 4 is moved forward in the extending direction J with respect to the tube 3.

More specifically, the bent portions of the claw portions 10b slip toward the opening 15a while coming into contact with a slope of the through-hole 15 with pressure and the arm portions 10a are pressed against an opening end 15bt on a proximal end side in the extending direction J of the opening 15b according to whether the tube 3 is moved backward in the extending direction J with respect to the specimen sampling tool 4 or the specimen sampling tool 4 is moved forward in the extending direction J with respect to the tube 3. Further, as shown in FIG. 3, the bent portions of the claw portions 10b and a distal end side in the extending direction J of the arm portions 10a are pressed against an opening end 15at on a distal end side in the extending direction J of the opening 15a, whereby the claw portions 10b are expanded in diameter to the distal end side in the extending direction J of the arm portions 10a and further to the outer side in the radial direction R than the outer peripheral surface 3g of the tube 3 from the through-hole 15.

That is, the distal end side in the extending direction J of the arm portions 10a and the claw portions 10b are physically expanded to the outer side in the radial direction R with the opening end 15at as a point of action by the slope of the through-hole 15 and the opening end 15at. Note that the diameter expansion of the claw portions 10b is recognized by an examiner under X-ray observation.

Therefore, the bent portions of the claw portions 10b come into contact with, with pressure, a part where the through-hole 15 is formed in the outer periphery of the tube 3. Therefore, it is desirable that the part is formed to be harder than other parts of the outer periphery of the tube 3 such that the part is not deformed by contact with the claw portions 10b and the claw portions 10b and the arm portions 10a can be physically expanded to the outer side in the radial direction R. For example, the part where the through-hole 15 is formed in the outer periphery of the tube 3 may be formed of metal.

As shown in FIG. 4, when an angle formed by a straight line a connecting the opening end 15bt on the proximal end side in the extending direction J of the opening 15b and the opening end 15at on the distal end side in the extending direction J of the opening 15a and the outer peripheral surface 3g of the tube 3 is represented as θ1 and an exterior angle in a crossing position of the straight line a and a straight line β perpendicular to the inner peripheral surface 14n of the subject and the outer peripheral surface 3g of the tube 3 is represented as 82, the through-hole 15 is formed at an inclination angle satisfying 90°+θ1≧θ2>θ1.

This is because, if the through-hole 15 is formed at the inclination angle satisfying 90°+θ1≧θ2>θ1, for example, even if the claw portions 10b are bent or folded at an angle 90° formed between the claw portions 10b and the arm portions 10a, since sharp edges 10bs of the distal ends of the claw portions 10b surely come into contact with the inner peripheral surface 14n (see FIG. 4) of the subject at an angle equal to or larger than 90°, cells or tissues on the inner peripheral surface 14n are easily shaved.

Note that, when the tube 3 is moved in the extending direction J relatively to the specimen sampling tool 4, as shown in FIG. 3, the ultrasound observation portion 4u completely projects further forward in the extending direction J than the opening 3k of the distal end 3s of the tube 3. Consequently, an observation of the examined region can be performed by the ultrasound observation portion 4u.

As shown in FIG. 3, in a state in which the claw portions 10b are expanded in diameter further to the outer side in the radial direction R than the outer peripheral surface 3g of the tube 3 from the through-hole 15 and the edges 10bs at the distal ends of the claw portions 10b come into contact with the inner peripheral surface 14n of the subject at an angle equal to or larger than 90°, the specimen sampling tool 4 and the tube 3 are integrally repeatedly moved back and forth in the extending direction J under the observation by the ultrasound observation portion 4u, whereby the distal ends shave cells or tissues on the inner peripheral surface 14n. Note that the cells or the tissues shaved from the inner peripheral surface 14n by the edges 10bs of the distal ends of the claw portions 10b are loaded on the loading surfaces 10cm of the loading portions 10c provided in the bent portions in the claw portions 10b.

After the diameter expansion of the claw portions 10b shown in FIG. 3, the claw portions 10b are retracted in the tube 3 according to relative movement of the specimen sampling tool 4 and the tube 3 opposite to the relative movement in the diameter expansion, more specifically, whether the tube 3 is moved forward in the extending direction J with respect to the specimen sampling tool 4 or the specimen sampling tool 4 is moved backward in the extending direction J with respect to the tube 3. More specifically, as shown in FIG. 2, the claw portions 10b are retracted in the through-hole 15 via the opening 15a. That is, the cells or the tissues loaded on the loading surfaces 10cm of the loading portions 10c are retracted in the through-hole 15.

Note that, as shown in FIG. 2, after the claw portions 10b are retracted in the through-hole 15, the distal end side in the extending direction J of the tube 3 is located to overlap the ultrasound observation portion 4u in the extending direction J. Therefore, since the distal end 3s of the tube 3 is observed by the ultrasound observation portion 4u, the examiner can easily recognize completion of the housing of the claw portions 10b in the through-hole 15 from a below-mentioned monitor 24 (see FIG. 14).

Conversely, when the distal end 3s of the tube 3 cannot be checked by the ultrasound observation portion 4u, the examiner can easily recognize from the monitor 24 (see FIG. 14) that the claw portions 10b have not been retracted in the through-hole 15 yet.

In a retracted state in the through-hole 15, most part of the opening 15a is closed by the bent portions of the claw portions 10b. Therefore, the cells or the tissues on the loading portions 10c less easily drop into the subject via the opening 15a.

Note that, after the retracting of the claw portions 10b in the through-hole 15, even if it is attempted to move the tube 3 forward in the extending direction J with respect to the specimen sampling tool 4 or move the specimen sampling tool 4 backward in the extending direction J with respect to the tube 3, the edges 10bs of the distal ends of the claw portions 10b are caught by an inclined surface of the through-hole 15. Therefore, the claw portions 10b do not enter the tube 3.

Next, action in the present embodiment is briefly explained.

When it is desired to sample cells or tissues of a small-diameter duct 14 (see FIG. 14) into which an insertion portion 22a of an endoscope 22 (see FIG. 14 for both of the insertion portion 22a and the endoscope 22) cannot be inserted, for example, an examined region of a lung periphery, first, the insertion portion 22a of the endoscope 22 is inserted from a mouth of the subject into a bronchus of a lung as deep as possible.

Subsequently, the treatment instrument for an endoscope 1 inserted into a treatment instrument channel of the endoscope 22 is projected forward from a distal end of the treatment instrument channel and further inserted to a periphery of the bronchus. The treatment instrument for an endoscope 1 is inserted until a distal end in the extending direction J of the treatment instrument for an endoscope 1 is located in the vicinity of the examined region of the lung periphery under X-ray observation.

Note that, when it is difficult to insert the treatment instrument for an endoscope 1 into the lung periphery, it is also possible that, first, a guide smaller in diameter than the treatment instrument for an endoscope 1 is inserted into the lung periphery via the treatment instrument channel and, thereafter, the treatment instrument for an endoscope 1 is inserted into the lung periphery via the guide.

A position of the tube 3 of the treatment instrument for an endoscope 1 can be always recognized under X-ray observation.

When the vicinity of the distal end of the treatment instrument for an endoscope 1 reaches the vicinity of a lesion of the lung periphery, the ultrasound transducer 4u is driven to move the treatment instrument for an endoscope 1 back and forth and check a position of the lesion while performing an ultrasound observation.

Thereafter, the tube 3 is moved backward in the extending direction J with respect to, for example, the specimen sampling tool 4. As a result, as shown in FIG. 3, the ultrasound observation portion 4u completely projects forward in the extending direction from the opening 3k of the distal end 3s of the tube 3. At the same time, the claw portions 10b are expanded in diameter further to the outer side in the radial direction R than the outer peripheral surface 3g of the tube 3 from the through-hole 15 via the opening 15a. Note that the diameter expansion of the claw portions 10b is checked by the examiner under X-ray observation.

Subsequently, under an observation of the ultrasound observation portion 4u, the specimen sampling tool 4 and the tube 3 are moved forward or backward in the extending direction J. After, for example, the lesion is accurately grasped as an examined region of the lung periphery, in a state in which the edges 10bs of the distal ends of the claw portions 10b are in contact with the inner peripheral surface 14n, the specimen sampling tool 4 and the tube 3 are repeatedly moved back and forth in the extending direction J, whereby cells or tissues on the inner peripheral surface 14n are shaved by the edges 10bs of the distal ends of the claw portions 10b. At this point, the examined region is shaved by the claw portions 10b under the observation by the ultrasound observation portion 4u. Therefore, it is possible to surely shave cells or tissues in the examined region with high position accuracy without mistaking the examined region.

Note that the cells or the tissues shaved by the edges 10bs of the distal ends of the claw portions 10b are loaded on the loading surfaces 10cm of the loading portions 10c provided in the bent portion in the claw portions 10b. Thereafter, the tube 3 is moved forward in the extending direction J with respect to the specimen sampling tool 4, whereby the claw portions 10b are retracted in the through-hole 15 via the opening 15a. That is, the cells or the tissues loaded on the loading surfaces 10cm of the loading portions 10c are retracted in the through-hole 15.

Finally, the treatment instrument for an endoscope 1 is pulled out via the treatment instrument channel of the endoscope 22. The tube 3 is moved backward in the extending direction J with respect to the specimen sampling tool 4 again and the claw portions 10b are expanded in diameter further to the outer side in the radial direction R than the outer peripheral surface 3g of the tube 3 from the through-hole 15 via the opening 15a, whereby the cells or the tissues of the examined region loaded in the through-hole 15 can be easily collected.

Note that the above is the same when the small-diameter duct 14 is a bile duct or a pancreatic duct.

As explained above, in the present embodiment, it is explained that the specimen sampling tool 4 is inserted in the tube 3 to be capable of moving back and forth in the extending direction J. It is explained that the proximal ends of the arm portions 10a in the rod for cell samplings 10, in which the claw portions 10b that shave biological tissues are provided at the distal ends of the arm portions 10a and the loading portions 10c on which shaved biological tissues are loaded are provided in the bent portions of the claw portions 10b, is fixed to the specimen sampling tool 4. Further, it is explained that the claw portions 10b are capable of being expanded in diameter further to the outer side in the radial direction R than the outer peripheral surface 3g of the tube 3 and capable of being retracted in the through-hole 15 formed in the tube 3 according to relative movement of the tube 3 and the specimen sampling tool 4 in the extending direction J.

It is explained that, after the diameter expansion of the claw portions 10b, the edges 10bs of the distal ends of the claw portions 10b come into contact with the inner peripheral surface 14n of the examined region at an angle equal to or larger than 90°.

Further, it is explained that, after the diameter expansion of the claw portions 10b, the tube 3 and the specimen sampling tool 4 are integrally moved back and forth in the extending direction in a state in which the edges 10bs of the distal ends of the claw portions 10b are in contact with the inner peripheral surface 14n, whereby cells or tissues are shaved and the shaved cells or tissues are loaded on the loading surfaces 10cm of the loading portions 10c.

Consequently, the edges 10bs of the distal ends of the claw portions 10b shave cells or tissues in a state in which the edges 10bs are in contact with the inner peripheral surface 14n of the examined region at an angle equal to or larger than 90°. As a result, the edges 10bs of the distal ends of the claw portions 10b can shave cells or tissues in a state in which the edges 10bs are deeply stuck in the examined region. Therefore, it is possible to shave the cells or the tissues more than the conventional specimen sampling tool such as a brush.

The cells or the tissues shaved by the claw portions 10b are loaded on the loading surfaces 10cm of the loading portions 10c without dropping into the subject. After the claw portions 10b are retracted in the through-hole 15, the cells or the tissues are surely loaded in the through-hole 15 in a state in which the opening 15a of the through-hole 15 is closed by the bent portions of the claw portions 10b. Therefore, the cells or the tissues do not drop into the subject via the opening 15a.

When cells or tissues are shaved by the claw portions 10b, the shaving is performed under the observation by the ultrasound observation portion 4u of the specimen sampling tool 4. Therefore, it is possible to accurately sample cells or tissues of the examined region with high position accuracy.

A reason for the above is as explained below. Conventionally, under X-ray observation, first, the tube with the X-ray marker provided at the distal end in the extending direction is inserted into the small-diameter duct 14, in a state in which the distal end of the tube is located in the vicinity of the examined region, the ultrasound probe is inserted into the tube and an accurate position of the examined region is checked, and, thereafter, the ultrasound probe is pulled out from the tube, the specimen sampling tool such as the brush is inserted into the tube again, and sampling of cells or tissues is performed. However, under X-ray observation, a slight movement of the tube cannot be detected and, in addition, cells or tissues can not be sampled under the observation by the ultrasound probe. Therefore, after the ultrasound probe is pulled out from the tube, the position of the examined region deviates because of insertion work for the specimen sampling tool, pulmonary respiration, and the like in addition to this pull-out work. Therefore, it is likely that cells or tissues are sampled in a position greatly different from the examined region in the extending direction J. However, in this configuration, since cells or tissues can be sampled under the observation by the ultrasound probe, it is possible to accurately sample cells or tissues of the examined region.

Further, the replacement work for the ultrasound probe and the specimen sampling tool in the tube 3 in the past is unnecessary. Therefore, it is possible to simplify a manipulation for cell or tissue sampling.

Consequently, it is possible to provide the treatment instrument for an endoscope 1 including the specimen sampling tool 4 that can sample cells or tissues on the inner peripheral surface 14n in the small-diameter duct 14 more than in the past and including the configuration capable of surely collecting the cells or the tissues sampled by the specimen sampling tool 4 in the tube 3.

Certainty of a diagnosis of cells or tissues is improved. A sampling time for cells or tissues can be reduced. Further, it is unnecessary to perform re-examination in order to sample cells or tissues. Therefore, it is possible to reduce burdens on both of the examiner and the subject.

Note that in the present embodiment explained above, modifications of which are explained below, it is explained that the specimen sampling tool 4 is configured with the mechanical radial scanning probe. However, the specimen sampling tool 4 is not limited to this. Naturally, the specimen sampling tool 4 may be configured with a radial electronic scanning probe.

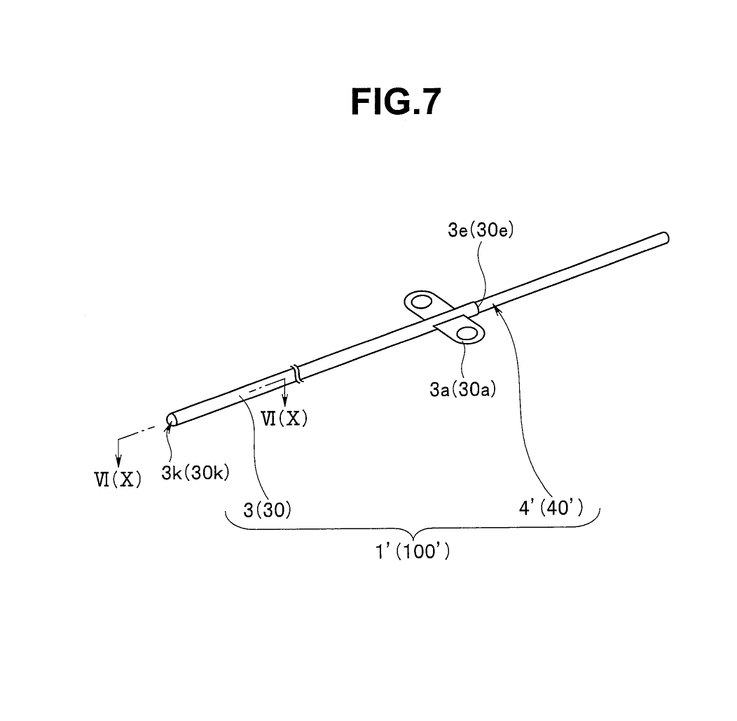

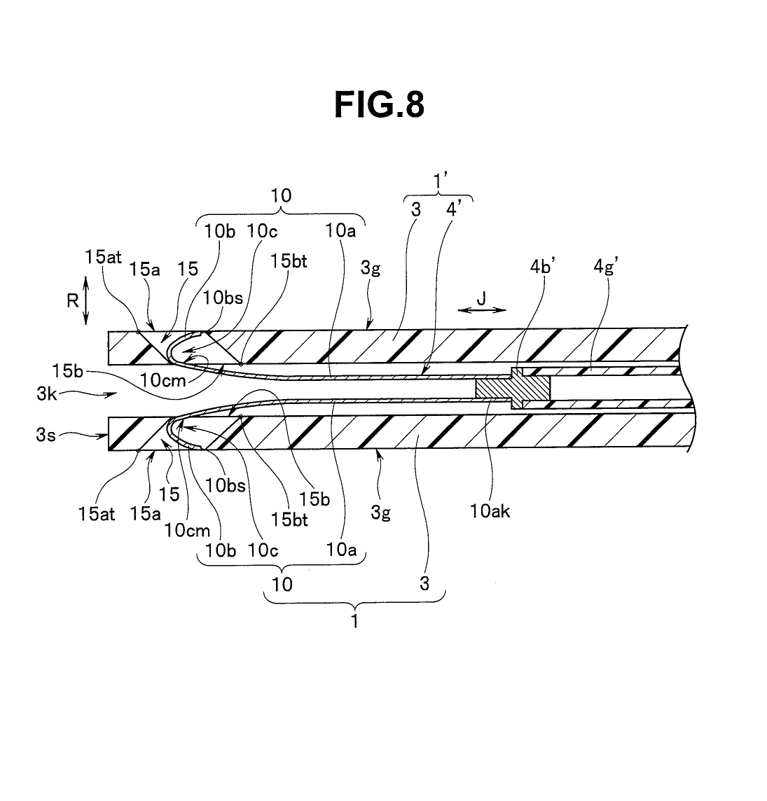

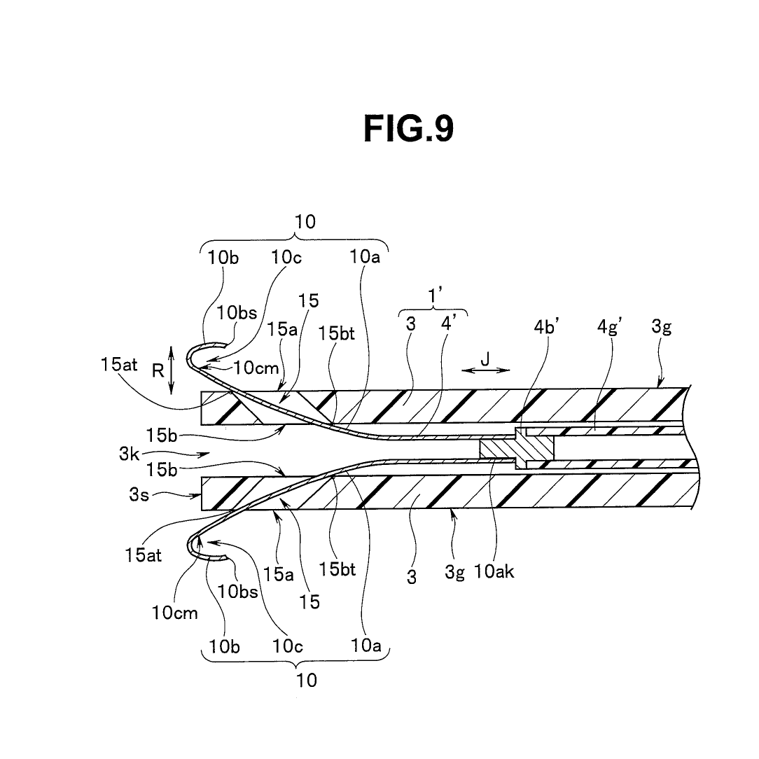

The modifications are explained below with reference to FIGS. 7 to 9. FIG. 7 is a perspective view showing a modification of the treatment instrument for an endoscope shown in FIG. 1. FIG. 8 is a partial sectional view of the treatment instrument for an endoscope taken along line VI-VI in FIG. 7. FIG. 9 is a partial sectional view showing a state in which arm portions of a rod for cell sampling in a specimen sampling tool shown in FIG. 8 are further expanded in diameter than a tube.

In the present embodiment explained above, the specimen sampling tool 4 is explained with reference to the ultrasound probe as an example. However, the specimen sampling tool 4 is not limited to this. The specimen sampling tool may be a member elongated in the extending direction J including the rod for cell sampling 10 and having no observation function.

More specifically, as shown in FIGS. 7 and 8, a main part of a treatment instrument for an endoscope 1′ includes the tube 3 and a specimen sampling tool 4′.

As shown in FIGS. 7 to 9, a main part of the specimen sampling tool 4′ includes, in the tube 3, an inner tube 4g′ extending further backward than a proximal end 3e in the extending direction J of the tube 3, a cap 4b′, a proximal end side in the extending direction J of which is fixed to a distal end in the extending direction J of the inner tube 4g′, and the rod for cell sampling 10, the proximal ends 10ak of the arm portions 10a of which are fixed to a distal end side in the extending direction J of the cap 4b′, extending forward in the extending direction J from the cap 4b′ in the tube 3.

Note that, also in the treatment instrument for an endoscope 1′, according to relative movement in the extending direction J of the tube 3 and the specimen sampling tool 4, as shown in FIG. 9, at least the claw portions 10b are capable of being expanded in diameter further to the outer side in the radial direction R than the outer peripheral surface 3g of the tube 3 from the through-hole 15 and, as shown in FIG. 8, the claw portions 10b are capable of being retracted in the through-hole 15.

The other components and action other than observation performed using the ultrasound probe are the same as those in the present embodiment explained above.

With such a configuration of the treatment instrument for an endoscope 1′, although cells or tissues cannot be sampled under an ultrasound observation, cells or tissues can be shaved more by the claw portions 10b than the conventional specimen sampling tool such as a brush as in the present embodiment explained above. Besides, the shaved cells or tissues can be surely collected by the loading portions 10c and the through-hole 15. Note that other effects are the same as those in the present embodiment explained above.

### Second Embodiment

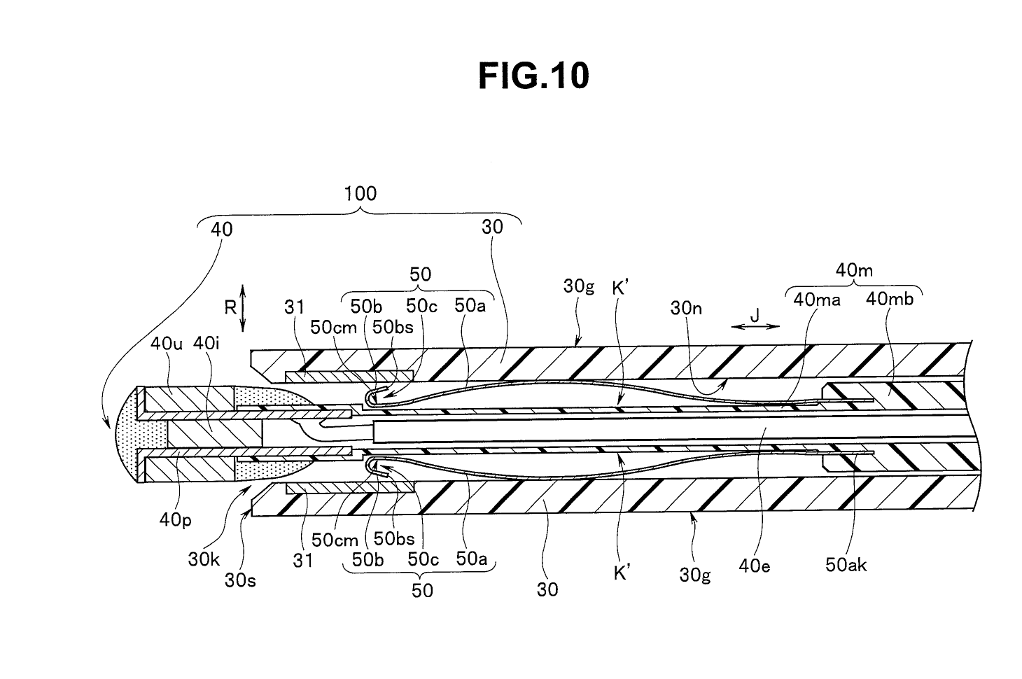

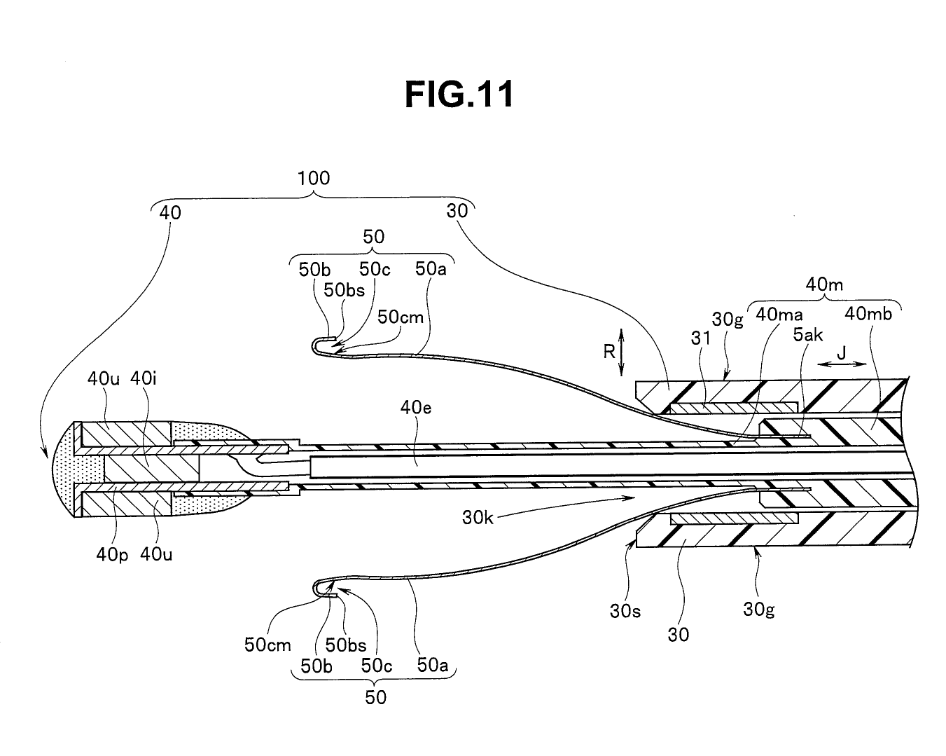

FIG. 10 is a partial sectional view of a treatment instrument for an endoscope in the present embodiment taken along line VIII-VIII in FIG. 1. FIG. 11 is a partial sectional view showing a state in which arm portions of a rod for cell sampling in a specimen sampling tool shown in FIG. 10 are further expanded in diameter than a tube.

Compared with the treatment instrument for an endoscope 1 in the first embodiment shown in FIGS. 1 to 4, a configuration of the treatment instrument for an endoscope in the second embodiment is different in that a claw portion of the rod for cell sampling is expanded in diameter further to an outer side in a radial direction than an outer peripheral surface of the tube via an opening at a distal end of the tube according to relative movement of the tube and the specimen sampling tool. Therefore, components same as the components in the first embodiment are denoted by the same reference numerals and signs and explanation of the components is omitted.

As shown in FIGS. 1 and 10, a main part of a treatment instrument for an endoscope 100 includes a tube 30 and a specimen sampling tool 40.

The tube 30 is formed to be elongated along the extending direction J of the specimen sampling tool 40. As shown in FIGS. 1 and 10, the specimen sampling tool 40 is capable of moving back and forth in the extending direction J on an inside of the tube 30. Note that a material of the tube 30 is the same as the material of the tube 3.

As shown in FIG. 1, an operation portion 30a for rotating the tube 30 in a circumferential direction of the tube 30 and moving the tube 30 back and forth in the extending direction J is provided at the proximal end side in the extending direction of the tube 30.

Further, as shown in FIG. 10, a metal ring 31 is fixed to an inner peripheral surface on a distal end side in the extending direction J in an outer peripheral part of the tube 30 by press fitting or integral machining with the tube 30. A distal end 30s in the extending direction J of the tube 30 is chamfered.

In the present embodiment, the specimen sampling tool 40 is configured with an ultrasound probe elongated along the extending direction J.

The specimen sampling tool 40 is configured with a radial electronic scanning probe. More specifically, the specimen sampling tool 40 includes an ultrasound observation portion 40u located at a distal end in the extending direction J and configured with a ring-like ultrasound transducer and a cylindrical flexible substrate 40p, on an outer peripheral surface on a distal end side in the extending direction J of which the ultrasound observation portion 40u is mounted. Further, the specimen sampling tool 40 includes an integrated circuit element 40i mounted on an inner peripheral surface of the flexible substrate 40p and configured to process ultrasound signals transmitted and received by the ultrasound observation portion 40u and an ultrasound transducer cable 40e, a distal end in the extending direction J of which is electrically connected to the flexible substrate 40p, extended to the connector 4x (see FIG. 1) and configured to transmit an electric pulse signal received from the below-mentioned ultrasound observation device 23 (see FIG. 14) to the ultrasound observation portion 40u.

The specimen sampling tool 40 includes a probe insertion portion 40m that covers an outer circumference of the ultrasound transducer cable 4e and a distal end in the extending direction of which is fixed to an outer peripheral surface on a proximal end side in the extending direction of the flexible substrate 40p.

A main part of the probe insertion portion 40m includes a large-diameter portion 40mb and a small-diameter portion 40ma located further on a distal end side in the extending direction J than the large-diameter portion 40b and configured to cover a distal end side in the extending direction J of the ultrasound transducer cable 40e. The specimen sampling tool 40 includes a rod for cell sampling 50.

That is, a main part of the specimen sampling tool 40 includes the ultrasound observation portion 40u, the flexible substrate 40p, the integrated circuit element 40i, the ultrasound transducer cable 40e, the probe insertion portion 40m, and the rod for cell sampling 50. The specimen sampling tool 40 is detachably attachable to the below-mentioned ultrasound observation device 23 (see FIG. 14) via the connector 4x provided at a proximal end in the extending direction J.

In the specimen sampling tool 40, according to relative movement of the tube 30 and the specimen sampling tool 40 in the extending direction J, at least the ultrasound observation portion 40u is capable of projecting further forward in the extending direction J than the distal end 30s via an opening 30k formed at the distal end 30s in the extending direction J of the tube 30.

A main part of the rod for cell sampling 50 includes at least two arm portions 50a having flexibility and located along the extending direction J, claw portions 50b provided at distal ends in the extending direction J of the respective arm portions 50a, distal ends of the claw portions 50b shaving cells or tissues on the inner peripheral surface 14n (see FIG. 4) of a tubular subject, and loading portions 50c provided in bent portions in the claw portions 50b, cells or tissues, which are collected specimens shaved by the claw portions 50b, being loaded on the loading portions 50c. In the rod for cell sampling 50, proximal ends 50ak in the extending direction J of the arm portions 50a are bound and fixed to a vicinity of a boundary between the large-diameter portion 40mb and the small-diameter portion 40ma of the probe insertion portion 40m by any one of bonding, insertion, and the like.

The arm portions 50a of the rod for cell sampling 50 is configured with a member having elasticity, for example, stainless steel for spring, a nickel titanium alloy, a cobalt chrome alloy, or the like. Further, as shown in FIG. 11, in a natural state, the arm portions 50a is formed such that a diameter in the radial direction R between the at least two claw portions 50b is larger than a diameter in the radial direction R of the tube 30. The arm portions 50a are reduced in diameter against the elasticity in the radial direction R and retracted in the tube 30.

In the arm portions 50a, as shown in FIG. 10, in a state in which the arm portions 50a are reduced in diameter and retracted in the tube 30, the claw portions 50b and a proximal end side in the extending direction J of the arm portions 50a are in non-contact with an inner peripheral surface 30n in an outer peripheral part of the tube 30. The other parts are retracted while having a convex shape on an outer side in the radial direction R that is in contact with the inner peripheral surface 30n.

Note that, in the retracted state, the rod for cell sampling 50 is located in a space K, which is formed because the small-diameter portion 40ma is smaller in diameter in the radial direction R than the large-diameter portion 40mb, along a vicinity of an outer periphery of the small-diameter portion 40m a.

Note that configurations and action of the claw portions 50b and the loading portions 50c are the same as those of the claw portions 10b and the loading portions 10c in the first embodiment. Therefore, explanation of the configurations and the action is omitted.

In the present embodiment, the claw portions 50b are capable of being retracted in the tube 30 from an outside of the tube 30 via the opening 30k of the distal end 30s in the extending direction J of the tube 30.

The claw portions 50b are expanded in diameter further to the outer side in the radial direction R than an outer peripheral surface 30g of the tube 30 according to relative movement of the specimen sampling tool 40 and the tube 30 in the extending direction J, that is, whether the tube 30 is moved backward in the extending direction J with respect to the specimen sampling tool 40 or the specimen sampling tool 40 is moved forward in the extending direction J with respect to the tube 30.

More specifically, the claw portions 50b together with a distal end side in the extending direction J of the arm portions 50a are expanded in diameter further to the outer side in the radial direction R than the outer peripheral surface 30g of the tube 30 by an elastic restoration force of the arm portions 50a via the opening 30k according to whether the tube 30 is moved backward in the extending direction J with respect to the specimen sampling tool 40 or the specimen sampling tool 40 is moved forward in the extending direction J with respect to the tube 30. Note that the diameter expansion of the claw portions 50b is checked by an examiner under X-ray observation.

When the tube 30 is moved in the extending direction J relatively to the specimen sampling tool 40, as shown in FIG. 11, the ultrasound observation portion 40u completely projects further forward in the extending direction J than the opening 30k of the distal end 30s of the tube 30. Consequently, the observation of the examined region can be performed by the ultrasound observation portion 40u.

As shown in FIG. 11, in a state in which the claw portions 50b are expanded in diameter further to the outer side in the radial direction R than the outer peripheral surface 30g of the tube 30 from the opening 30k and edges 50bs at the distal ends of the claw portions 50b come into contact with the inner peripheral surface 14n of the subject at an angle equal to or larger than 90°, the specimen sampling tool 40 and the tube 30 are integrally repeatedly moved back and forth in the extending direction J under the observation by the ultrasound observation portion 40u, whereby the distal ends shave cells or tissues on the inner peripheral surface 14n. Note that the cells or the tissues shaved from the inner peripheral surface 14n by the edges 50bs of the distal ends of the claw portions 50b are retracted on the loading surfaces 50cm of the loading portions 50c provided in the bent portions in the claw portions 50b.

After the diameter expansion of the claw portions 50b shown in FIG. 11, the claw portions 50b are reduced in diameter and retracted in the tube 30 via the opening 30k as shown in FIG. 10 according to relative movement of the specimen sampling tool 40 and the tube 30 opposite to the relative movement in the diameter expansion, more specifically, whether the tube 30 is moved forward in the extending direction J with respect to the specimen sampling tool 40 or the specimen sampling tool 40 is moved backward in the extending direction J with respect to the tube 30.

At this point, since the distal end 30s of the tube 30 is chamfered and a taper surface is formed at the distal end 30s, the claw portions 50b are not caught by the distal end 30s. The claw portions 50b are guided by the taper surface and smoothly retracted in the tube 30.

As explained above, the arm portions 50a has a shape in which the claw portions 50b and the proximal end side in the extending direction J of the arm portions 50a are non-contact with the inner peripheral surface 30n in the outer peripheral part of the tube 30 and the other parts are retracted while having the convex shape on the outer side in the radial direction R that is in contact with the inner peripheral surface 30n. Consequently, as shown in FIG. 10, after the claw portions 50b are retracted in the tube 30, when the rod for cell sampling 50 is retracted in the tube 30 via the opening 30k, only the proximal end side in the extending direction J of the arm portions 50a comes into contact with the distal end 30s of the tube 30, that is, the claw portions 50b do not come into contact with the distal end 30s of the tube 30. Therefore, the sampled cells or tissues are not caught by the distal end 30s of the tube 30.

In this retracted state, most part of the opening 30k is closed by the ultrasound observation portion 40u. Therefore, the cells or the tissues on the loading portions 50c less easily drop into the subject via the opening 30k.

Note that, after the retracting of the claw portions 50b in the tube 30, it is possible to easily collect the cells or the tissues by pulling out the specimen sampling tool 40 from the tube 30.

When the treatment instrument for an endoscope 100 has such a configuration, effects same as the effects in the first embodiment can be obtained. Further, since the specimen sampling tool 40 can be removed in a state in which the tube 30 is stored in the subject, it is possible to repeatedly perform cell sampling. It is possible to insert a specimen sampling tool such as a brush, a puncture needle, or forceps into the tube 30 instead of the specimen sampling tool 40 to perform sampling of cells or tissues. It is possible to easily perform a treatment manipulation such as radio wave cauterization following the sampling of cells or tissues.

Note that, in the present embodiment explained above, modifications of which are explained below, it is explained that the specimen sampling tool 40 is configured with the radial electronic scanning probe. However, the specimen sampling tool 40 is not limited to this. Naturally, the specimen sampling tool 40 may be configured with a mechanical radial scanning probe.

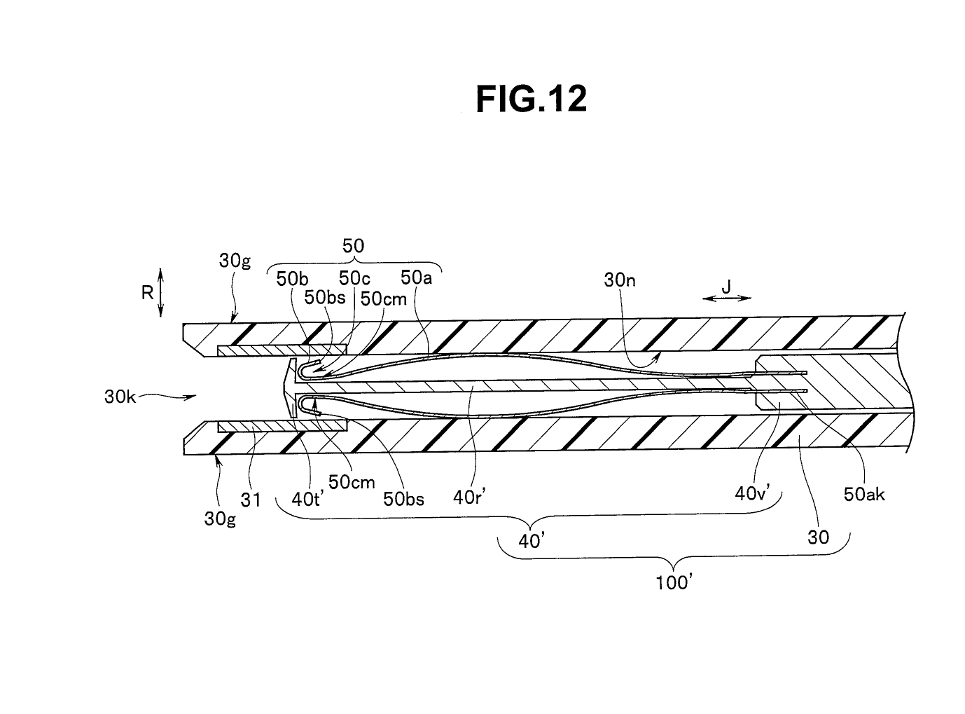

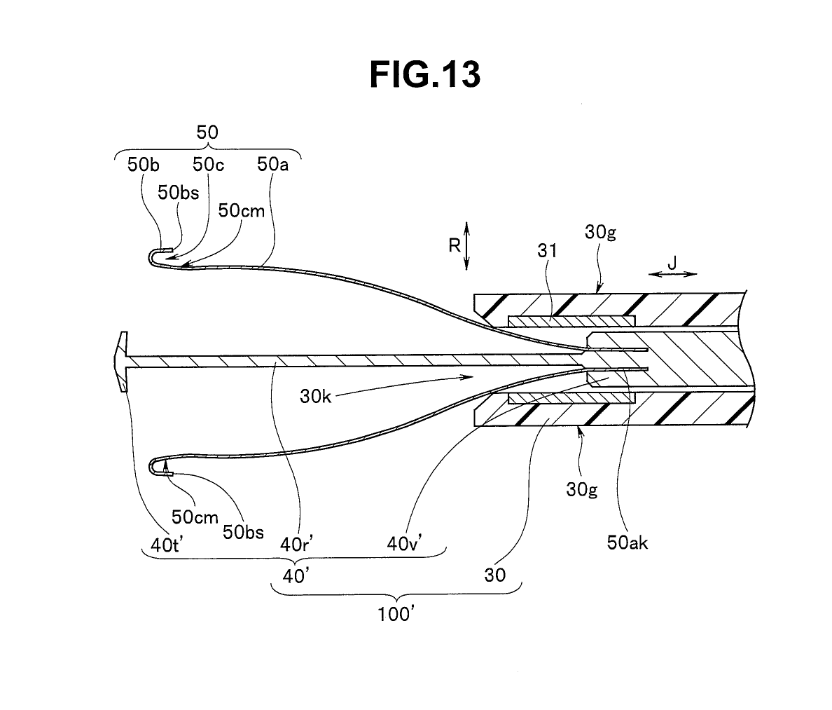

The modifications are explained below with reference to FIGS. 12 and 13. FIG. 12 is a partial sectional view of the treatment instrument for an endoscope taken along line X-X in FIG. 7. FIG. 13 is a partial sectional view showing a state in which the arm portions of the rod for cell sampling in the specimen sampling tool shown in FIG. 12 is further expanded in diameter than the tube.

In the present embodiment explained above, it is explained that the specimen sampling tool 40 is the ultrasound probe. However, the specimen sampling tool 40 is not limited to this. The specimen sampling tool may be a member elongated in the extending direction J including the rod for cell sampling and having no observation function.

More specifically, as shown in FIGS. 12 and 13, a main part of a treatment instrument for an endoscope 100′ includes the tube 30 and a specimen sampling tool 40′.

As shown in FIGS. 12 and 13, a main part of the specimen sampling tool 40′ includes, in the tube 30, a probe insertion portion large-diameter portion 40v′ extending further backward than a proximal end 30e in the extending direction J of the tube 30, a probe insertion portion small-diameter portion 40r′ projecting forward in the extending direction J from the probe insertion portion large-diameter portion 40v′, a brim portion 40t′ provided at a distal end in the extending direction J of the probe insertion portion small-diameter portion 40r′, and the rod for cell sampling 50.

Note that, the proximal ends 50ak of the arm portions 50a of the rod for cell sampling 50 are fixed to a vicinity of a boundary between the probe insertion portion large-diameter portion 40v′ and the probe insertion portion small-diameter portion 40r′ by any one of bonding, insertion, and the like. Therefore, in the rod for cell sampling 50, the claw portions 50b are located further on the proximal end side in the extending direction J than brim portions 50t and the arm portions 50a are located along an outer periphery of the probe insertion portion small-diameter portion 40r′.

Note that, also in the treatment instrument for an endoscope 100′, according to relative movement in the extending direction J of the tube 30 and the specimen sampling tool 40′, as shown in FIG. 13, the claw portions 50b together with the distal end side in the extending direction J of the arm portions 50a are capable of being expanded in diameter further to the outer side in the radial direction R than the outer peripheral surface 30g of the tube 30 from the opening 30k and, as shown in FIG. 12, the claw portions 50b are capable of being retracted in the tube 30 via the opening 30k.

After the sampling of the cells or the tissues, as in the present embodiment explained above, the cells or the tissues loaded on the loading surfaces 50cm of the loading portions 50c are retracted in the tube 30. At this point, the brim portion 40t′ prevents the cells or the tissues from dropping into the subject via the opening 30k. Therefore, the brim portion 40t′ only has to be provided according to necessity and is not an essential component.

The other components and action other than observation performed using the ultrasound probe are the same as those in the present embodiment explained above.

With such a configuration of the treatment instrument for an endoscope 100′, although cells or tissues cannot be sampled under an ultrasound observation, cells or tissues can be shaved more by the claw portions 50b than the conventional specimen sampling tool such as a brush as in the present embodiment explained above. Further, the shaved cells or tissues can be surely collected by the loading portions 50c and the opening 30k. Note that, other effects are the same as those in the present embodiment explained above.

In the examples explained in the first and second embodiments explained above, the number of the arm portions 10a and 50a of the rods for cell sampling 10 and 50 fixed to the specimen sampling tools 4, 4′, 40, and 40′ is two. However, the number of the arm portions 10a and 50a is not limited to this. It goes without saying that the arm portions 10a and 50a may be provided in any number.

As the number of the arm portions 10a and 50a is larger, it is less necessary to perform alignment for rotating the specimen sampling tools 4, 4′, 40, and 40′ in the circumferential direction with respect to the examined region and bringing the claw portions 10b and 50b into contact with the examined region. That is, it is easy to align the claw portions 10b and 50b with the examined region.

Note that the treatment instrument for an endoscope 1 in the first embodiment and the treatment instrument for an endoscope 100 in the second embodiment explained above are used for, for example, an endoscope system shown in FIG. 14.

FIG. 14 is a diagram schematically showing an endoscope system including the treatment instrument for an endoscope shown in FIG. 1.

As shown in FIG. 14, the endoscope 22 included in an endoscope system 200 includes the elongated endoscope insertion portion 22a having flexibility. An operation portion 22b is provided on an examiner side of the endoscope insertion portion 22a.

Further, a universal cord 22c is extended from the operation portion 22b. A scope connector 22d is provided at an end of the universal cord 22c. A video processor device and a light source device not shown in the figure are connected to the scope connector 22d.

A treatment instrument insertion port 22e is opened in the vicinity of a coupling portion of the endoscope insertion portion 22a and the operation portion 22b. The treatment instrument channel is inserted through the treatment instrument insertion port 22e. The treatment instrument channel is formed in the endoscope insertion portion 22a. A distal end of the treatment instrument channel is opened to a distal end face of the endoscope insertion portion 22a. The connector 4x provided at the proximal end of the specimen sampling tool 4 (40) of the treatment instrument for an endoscope 1 (100) is connected to a connector receiving portion 23a of the ultrasound observation device 23. The ultrasound observation device 23 causes the monitor 24 to display an ultrasound image obtained by the ultrasound observation portion 4u (40u).

What is claimed is:

1. A treatment instrument for an endoscope comprising:
a specimen sampling tool including at least two arm portions having flexibility, loading portions provided at a distal end in an extending direction of the arm portions, a collected specimen from a subject being loaded on the loading portion, and claw portions provided at the distal end of the arm portions, formed to be bent to a loading surface side of the loading portion, on which the collected specimen is loaded, to include the loading portion on an inside, and configured to shave the subject, a proximal end in the extending direction of the arm portions being bound and fixed to the specimen sampling tool; and
a tube on an inside of which the specimen sampling tool is placed to be capable of moving back and forth in the extending direction, wherein
at least the claw portions of the arm portions are expanded in diameter further to an outer side of the tube in a radial direction than the tube according to relative movement of the specimen sampling tool and the tube in the extending direction,
as a result of the diameter expansion, a plurality of the loading surfaces and a plurality of the claw portions respectively face outward with respect to an axis of the specimen sampling tool, and
after the diameter expansion, the arm portions are retracted in the tube according to relative movement of the specimen sampling tool and the tube opposite to the relative movement in the diameter expansion in the extending direction.

2. The treatment instrument for an endoscope according to claim 1, wherein
a through-hole is formed in an outer periphery part on a distal end side in the extending direction of the tube,
at least the claw portions of the arm portions are expanded in diameter further to an outer side of the tube in the radial direction than the tube via the through-hole according to the relative movement of the specimen sampling tool and the tube in the extending direction, and
after the diameter expansion, the arm portions are retracted in the through-hole according to the relative movement of the specimen sampling tool and the tube opposite to the relative movement in the diameter expansion in the extending direction.

3. The treatment instrument for an endoscope according to claim 2, wherein
the through-hole is formed to tilt such that an opening on an inner side in the radial direction is located further on a proximal end side in the extending direction than an opening on an outer side in the radial direction, and
at least the claw portions of the arm portions are expanded in diameter to the outer side in the radial direction by being pressed against an end of the opening on the outer side of the through-hole from the through-hole according to the relative movement of the specimen sampling tool and the tube in the extending direction.

4. The treatment instrument for an endoscope according to claim 2, wherein a part of the tube where the through-hole is formed is formed to be harder than other parts of the tube.

5. The treatment instrument for an endoscope according to claim 1, wherein the arm portions are configured of an elastic member, in which a diameter in
the radial direction between at least two of the claw portions after the diameter expansion is formed larger than a diameter of the tube, and are reduced in diameter against elasticity and stored in the tube,
at least the claw portions of the arm portions are expanded in diameter further to the outer side of the tube in the radial direction than the tube by an elastic restoration force of the arm portions via an opening at a distal end in the extending direction of the tube according to the relative movement of the specimen sampling tool and the tube in the extending direction, and
after the diameter expansion, the diameter of the arm portions are reduced and retracted in the tube via the opening according to the relative movement of the specimen sampling tool and the tube opposite to the relative movement in the diameter expansion in the extending direction.

6. The treatment instrument for an endoscope according to claim 5, wherein, the arm portions are in a diameter reduced state in the tube, the claw portions and a proximal end of the arm portions are in non-contact with an inner peripheral surface of the tube and other parts of the arm portions have a convex shape on an outer side in the radial direction that is in contact with the inner peripheral surface.

7. The treatment instrument for an endoscope according to claim 1, wherein
a distal end in the extending direction of the tube is opened, and
the specimen sampling tool is an ultrasound probe in which at least a part of an ultrasound observation portion is projected further forward in the extending direction than the distal end of the tube via the opening.

8. The treatment instrument for an endoscope according to claim 7, wherein, when at least the claw portions are expanded in diameter further to an outer side of the tube in the radial direction than the tube, the ultrasound observation portion completely projects further forward in the extending direction than the opening of the tube.

9. The treatment instrument for an endoscope according to claim 6, wherein a distal end side in the extending direction of the tube is located to overlap the ultrasound observation portion in the extending direction when the arm portions are retracted in the tube.
